(19) United States
(12) Patent Application Publication
(10) Pub. No.: US 2024/0099922 A1
(43) Pub. Date: Mar. 28, 2024

(54) SHOE BALANCING DEVICE AND METHOD

(71) Applicant: Amit HAVIV, Petah Tikva (IL)

(72) Inventor: Amit HAVIV, Petah Tikva (IL)

(21) Appl. No.: 18/276,134

(22) PCT Filed: Apr. 28, 2022
(86) PCT No.: PCT/IL2022/050433

§ 371 Date: Aug. 7, 2023

Related U.S. Application Data
(60) Provisional application No. 63/181,912, filed on Apr. 29, 2021.

(51) Int. Cl. A61H 3/00; A43B 3/34; A43B 7/00

(52) U.S. Cl. CPC A61H 3/00; A43B 3/34; A43B 7/00; A61H 2003/007; A61H 2201/123; A61H 2201/1642; A61H 2201/165; A61H 2201/5069; A61H 2201/5084; A61H 2230/625; G08B 21/02

(57) ABSTRACT

A shoe balancing device including a housing, configured to be attached to a shoe of a wearer; a power source to power the device; one or more sensors configured to determine an imbalance state of the device; a hardware board configured to act as the operation control center of the device; a motor configured to be activated by the hardware board if the one or more sensors determine(s) an imbalance state beyond a predetermined threshold; and a stabilization rod connected to the motor so as to be moveable from a default undeployed state to a deployed state, wherein the deployed state being a position in contact with a surface underneath the wearer's location, wherein when a wearer of the shoe begins to fall the device is actuated.

### TECHNOLOGICAL FIELD

The presently disclosed subject matter is in the field of falls prevention, in particular a device and method for balancing/stabilizing a person to help prevent falls.

### BACKGROUND

People may experience imbalance and consequently instability and falls for several reasons, such as obstacles, dizziness, weakness, muscle atrophy/sarcopenia, involuntary weight loss, impaired vision or hearing, medicinal side effects, and the like. Falls are most common in elderly population and can result in serious injuries.

When falling or losing balance, one's body may lean to one side (most commonly left or right), with most of one's body weight on one side of the foot. Fear of falling is in itself a risk factor for falling, aside from being unpleasant.

### SUMMARY OF THE INVENTION

The presently disclosed subject matter relates to a shoe balancing device and method, configured to mitigate falling, in particular falling to the left or right sides, and optionally rear falling.

According to one aspect of the presently disclosed subject matter there is provided a shoe balancing device comprising:
a housing configured to be attached to a shoe of a wearer;
a power source to power the device;
an accelerometer and/or gyroscope sensor configured to determine an imbalance state of the device;
a hardware board configured to act as the operation control center of the device;
a motor configured to be activated by the hardware board if the accelerometer and/or gyroscope sensor determines an imbalance state beyond a predetermined threshold; and
a stabilization rod operably connected to the motor so as to be moveable from a default undeployed state to a deployed state, the deployed state being a position in contact with a surface underneath the wearer's location, wherein when a wearer of the shoe begins to fall, the device is actuated.

In one or more embodiments, the device comprises straps to attach the housing to the shoe.

In one or more embodiments, the device further comprises a global positioning system (GPS) module to provide location information of the wearer.

In one or more embodiments, the motor is configured to move the stabilization rod downwardly toward the surface to thereby apply a resistance force thereto.

In one or more embodiments, the motor is configured to linearly move the stabilization rod.

In one or more embodiments, the device further comprises a solenoid pin configured to reversibly lock the stabilization rod in place after deployment thereof.

In one or more embodiments, the device further comprises a potentiometer to read values from the hardware board to provide an indication of battery power.

In one or more embodiments, the power source is a battery.

In one or more embodiments, the device is configured to be positioned on a right shoe, a left shoe, or both shoes.

In one or more embodiments, the device is configured to be positioned on the lateral facet of a wearer shoe.

In one or more embodiments, the device is configured to stabilize the wearer and prevent falling thereof.

In one or more embodiments, the device further comprises an alarm mechanism comprising a vibration and/or an audible warning to allow for an advance warning to the wearer about a potential fall.

In one or more embodiments, the stabilization rod is configured to revert to the undeployed state when the wearer is essentially vertically stabilized.

In one or more embodiments, the threshold is at least 20 degrees movement of the wearer toward the ground/floor.

In one or more embodiments, the threshold is at least 30 degrees movement of the wearer toward the ground/floor.

In one or more embodiments, the stabilization rod is operably connected to the motor via a stabilization rod connector.

In one or more embodiments, the connector is removably connected to the stabilization rod via.

In one or more embodiments, the attachment between the connector and the stabilization rod is via threads and corresponding internal threads located on the attachment ends of the stabilization rod and stabilization rod connector, respectively.

In one or more embodiments, the attachment between the connector and the stabilization rod is via threads and corresponding internal threads located on the attachment ends of the stabilization rod connector and stabilization rod, respectively.

In one or more embodiments, the stabilization rod outwardly protrudes from a side wall of the housing when the device is actuated.

In one or more embodiments, the stabilization rod outwardly protrudes from a rear wall of the housing when the device is actuated.

According to one aspect of the presently disclosed subject matter there is provided a method of preventing or indicating a fall of a wearer of a shoe balancing device connected to a shoe of the wearer upon a surface. The method includes the steps of
determining if the shoe balancing device is at an angle beyond a predetermined threshold; and
activating a stabilizing rod, if said threshold is surpassed, to move from a non-deployed default state to a deployed state, wherein a free end of the stabilizing rod is moved to a position that interfaces with the surface underneath the wearer's location.

In one or more embodiments, the stabilizing rod is linearly moved by the rod toward the ground/floor to stabilize the wearer.

In one or more embodiments, if the threshold is surpassed by a predetermined period of time, a signal is sent to a telephone of another person or to a monitoring service.

In one or more embodiments, the predetermined period of time is at least 10 seconds, at least 20 seconds, at least 30 seconds, at least 40 second, at least 50 seconds, or at least 60 seconds. Each possibility represents a separate embodiment of the invention.

In one or more embodiments, the predetermined period of time is no more than 60 seconds, no more than 50 seconds, no more than 40 seconds, no more than 30 seconds, no more than 20 seconds, or no more than 10 seconds. Each possibility represents a separate embodiment of the invention.

In one or more embodiments, the method comprises providing a location of the device using a GPS module.

In one or more embodiments, the threshold is at least 20 degrees movement of the wearer toward the ground/floor. In one or more embodiments, the threshold is at least 30 degrees movement of the wearer toward the ground/floor.

### BRIEF DESCRIPTION OF THE DRAWINGS

The presently disclosed subject matter may be more clearly understood upon reading of the following detailed description of non-limiting exemplary embodiments thereof, with reference to the following drawings, in which:

FIGS. 1 and 2 are perspective views of a shoe balancing device in accordance with one example of the presently disclosed subject matter, when attached to a shoe.

FIG. 3 are perspective views of components of a shoe balancing assembly of the herein shoe balancing device, in accordance with an example of the presently disclosed subject matter.

FIG. 4 is a schematic view of internal components of the shoe balancing device, in accordance with an example of the presently disclosed subject matter.

FIG. 5 is a perspective view of the medial/inner side of a shoe illustrating connector straps for the shoe balancing device in accordance with an example of the presently disclosed subject matter.

FIG. 6 is a perspective view of the shoe balancing device, in a deployed configuration, in accordance with an example of the presently disclosed subject matter.

FIG. 7 is a perspective view of the shoe balancing device, illustrating a rear stabilization rod to assist in preventing back falls, in accordance with an example of the presently disclosed subject matter.

FIG. 8 is a flow chart depicting scenarios of the shoe balancing device in operation, in accordance with an example of the presently disclosed subject matter.

The following detailed description of embodiments of the presently disclosed subject matter refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

### DETAILED DESCRIPTION

It is understood that the presently disclosed subject matter is not limited to the particular methodology, systems, devices, apparatus, items or products etc., described herein, as these may vary as the skilled artisan will recognize. It is also to be understood that the terminology used herein is used for the purpose of describing particular embodiments only and is not intended to limit the scope of the presently disclosed subject matter. The following exemplary embodiments may be described in the context of exemplary shoe balancing devices for ease of description and understanding. However, the presently disclosed subject matter is not limited to the specifically described products and methods and may be adapted to various applications without departing from the overall scope of the presently disclosed subject matter.

The present invention provides a shoe balancing device configured to protect a wearer from falling. The shoe balancing device includes a motorized stabilization rod that is movable between an undeployed state and a deployed state in accordance with monitored movements that potentially lead to falling. In the undeployed state, the stabilization rod does not touch the ground below the wearer. In the deployed state, the stabilization rod touches the ground below the wearer and balances the wearer, thus preventing a potentially detected fall.

Thus, according to an aspect of the presently disclosed subject matter, there is provided a shoe balancing device comprising:
a housing configured for attachment to a shoe of a wearer;
a power source to power the device;
an accelerometer and/or gyroscope sensor configured to determine an imbalanced state of the device;
a hardware board configured to act as an operation control center of the device;
a motor configured to be activated by the hardware board if the accelerometer and/or gyroscope sensor determines an imbalanced state beyond a predetermined threshold; and
a stabilization rod operably connected to the motor so as to be moveable from a default undeployed state to a deployed state, the deployed state being a position in contact with a surface where the wearer is located, wherein when a wearer of the shoe begins to fall the device is actuated.

According to another aspect of the presently disclosed subject matter, there is provided a shoe balancing device comprising:
a housing configured for attachment to a shoe of a wearer;
a power source to power the device;
sensing means configured to determine an imbalanced state of the device;
a hardware board configured to act as an operation control center of the device;
a motor configured to be activated by the hardware board if the sensing means determine an imbalanced state beyond a predetermined threshold; and
a stabilization rod operably connected to the motor so as to be moveable from a default undeployed state to a deployed state, the deployed state being a position in contact with a surface where the wearer is located, wherein when a wearer of the shoe begins to fall the device is actuated.

The housing of the herein shoe balancing device may feature various forms, including, without limitation a square shape, a rectangular shape, and a circular shape. The device (housing thereof) is configured for removable attachment to a shoe. The device may be attached to a left shoe, a right shoe, or each shoe may have a device attached thereto.

The housing may be attached to the right shoe. The housing may be attached to the left shoe. The housing may be attached to both the left shoe and the right shoe. Optionally, each shoe, right or left, is attached to the herein shoe balancing device, and each device orientationally fits its own shoe (right or left) such that the devices seem inverted with respect to each other and appear as a “mirror image”. To facilitate efficient prevention of falls, the housing is preferably attached to the lateral side (the external facet) of the shoe. Thus, the housing may be attached to the lateral side (the external facet) of the right shoe; to the lateral side (the external facet) of the left shoe; or the housing may be attached to the lateral side (the external facet) of both the right shoe and the left shoe.

The housing may be made from various materials or combination of materials, such as polymers, e.g., a rubber, a polycarbonate, and a metal, or metal alloys, and the like.

The housing features a stabilization rod which is actuated to move to the deployed state and thereby move downwardly when a fall is detected.

The stabilization rod is made from a rigid material to allow a stable and reliable support to the wearer and effectively prevent a fall. Exemplary rigid materials include, for example, rigid polymeric materials (e.g., polypropylene), metals (e.g., aluminum, iron), or metal alloys (e.g., steel). The rigid material may be coated with a rubber or the like to improve a stable interface between a free end of the stabilization rod and the floor/ground or any other surface underneath the wearer.

The stabilization rod may feature a linear movement. Optionally, the stabilization rod may feature a rotational movement, or a partially rotational movement. The linear movement from the undeployed state to the deployed state includes a linear movement downwards toward the ground. The movement from the deployed state to the undeployed state includes movement upward. The herein device may further comprise a locking pin, e.g., a solenoid pin, configured to reversibly lock the stabilization rod in place after deployment thereof.

Optionally, the herein housing includes a side stabilization rod to prevent side falls and also a rear stabilization rod to prevent backward falls. A side stabilization rod as well as a rear stabilization rod also can effectively prevent side-backward falls.

The device comprises at least one strap (e.g., lanyard) to attach the housing to the shoe. The straps may be made from various materials, e.g., a stretchy material, e.g., a stretchy fabric, such as spandex. The device may include two or more straps, at least three straps, or at least four straps. In an exemplary embodiment, the device includes two straps. The straps may include attachment mechanisms to attach the two ends thereof, such as buckles composed of male and female buckle portions.

The herein device may additionally include a global positioning system (GPS) module to provide location information of the wearer.

As used herein the term “GPS module” refers to processor(s) and antenna(s) that directly receive data sent by satellites through dedicated radio frequency (RF frequencies) to accurately calculate a position and time of the wearer.

The device further includes a hardware board (e.g., an open-source hardware board, such as an ESP32 hardware board) configured to act as the operation control center. The hardware board may include a single or a dual-core, and support Wi-Fi connectivity, support Bluetooth specifications, RAM (Random Access Memory), I2C (Inter-Integrated Circuit) Interface, and/or have programmable GPIOs (general-purpose input/output). The support with Wi-Fi and Bluetooth is configured to transfer falling data to a remote-control center (e.g., through the wearer's smartphone mobile software App (application)) when a fall incident is detected, optionally in conjunction with the operation of the stabilization rod.

The motor of the herein device may be any suitable motor capable of actuating the movement of the stabilization rod from the undeployed state to the deployed state and from the deployed state to the undeployed state. The motor is disposed within the housing of the device. The motor may be for example, a servo-motor.

As used herein the term “servo-motor” refers to an actuator (typically a rotary actuator or linear actuator) that allows for a precise control of movement of the stabilization rod from the undeployed state to a deployed state and/or from the deployed state to an undeployed state.

The motor is configured to be activated by the hardware board if the sensing means (accelerometer and/or gyroscope) determines there is an imbalance state beyond a predetermined threshold. The threshold may be a tilt of the shoe with respect to the ground of at least 10 degrees sideway. For example, the threshold may be of at least 15 degrees, the threshold may be of at least 20 degrees, at least 30 degrees, at least 40 degrees, at least 50 degrees, or at least 60 degrees sideways. Each possibility represents a separate embodiment of the invention. The threshold may be adapted to each of the right and left shoe such that in a device that is designed for the right shoe, the threshold will be a right tilt and in a device that is designed for the left shoe, the threshold will be a left tilt.

The motor may be actuated to move the stabilization rod by one or more sensors for position feedback. The sensor may be an accelerometer and a gyroscope sensor, e.g., a GY-521 MPU-6050 3 Axis gyroscope and 3 Axis Accelerometer Module. The sensor may be a proximity sensor. The sensor may be constituted by a single sensor, two sensors, three sensors, or more sensors. Each possibility represents a separate embodiment of the invention.

As used herein the term sensing means refers to devices that can sense and measure three-dimensional rotation, static acceleration due to gravity, as well as motion, inclination, shock, and/or dynamic acceleration due to vibration. The term includes accelerometer and a gyroscope.

The power source may be a battery, or a solar board, or any other suitable power source.

The herein device may further comprise a potentiometer to read values from the hardware board to provide an indication of battery power.

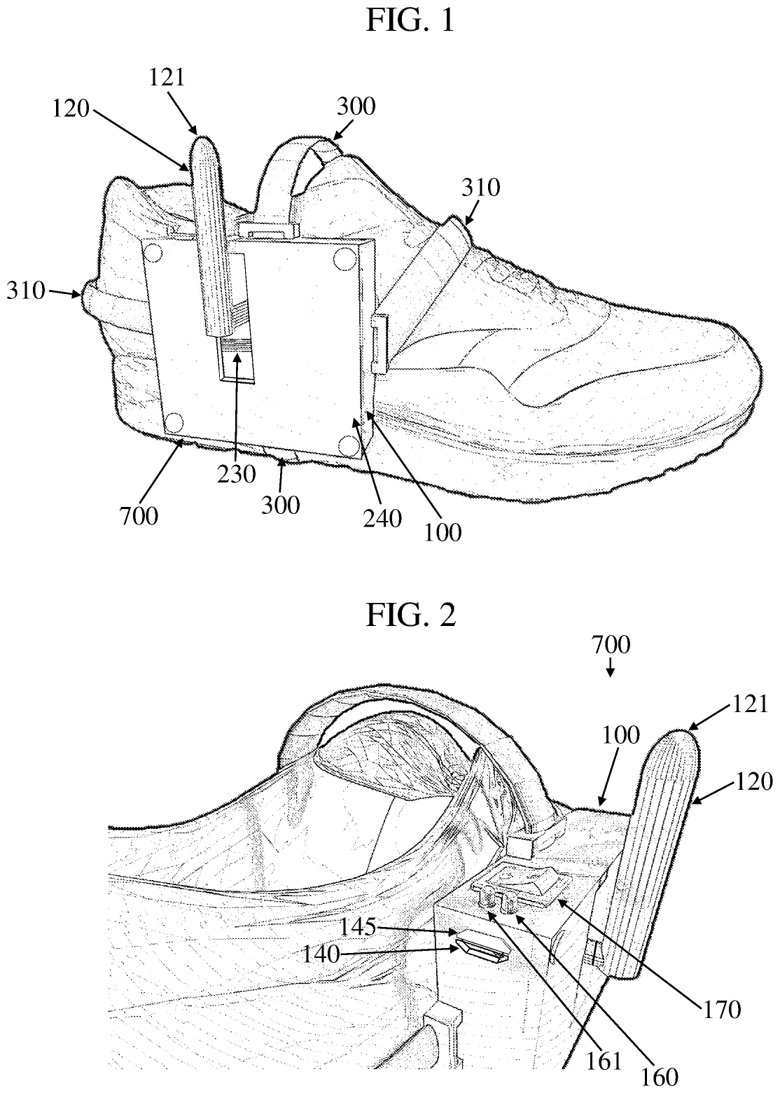

FIG. 1 and FIG. 2 show an example of a shoe balancing device 700 attached to the right side of a right shoe. An additional shoe balancing device can be attached to a left shoe, and the device can be considered as a pair, i.e., a device that fits the left shoe and a device that fits the right shoe. The shoe balancing device 700 can be positioned on either or both left and right shoes, when attached to the side facet(s) of the shoe, while facing the lateral side(s) thereof.

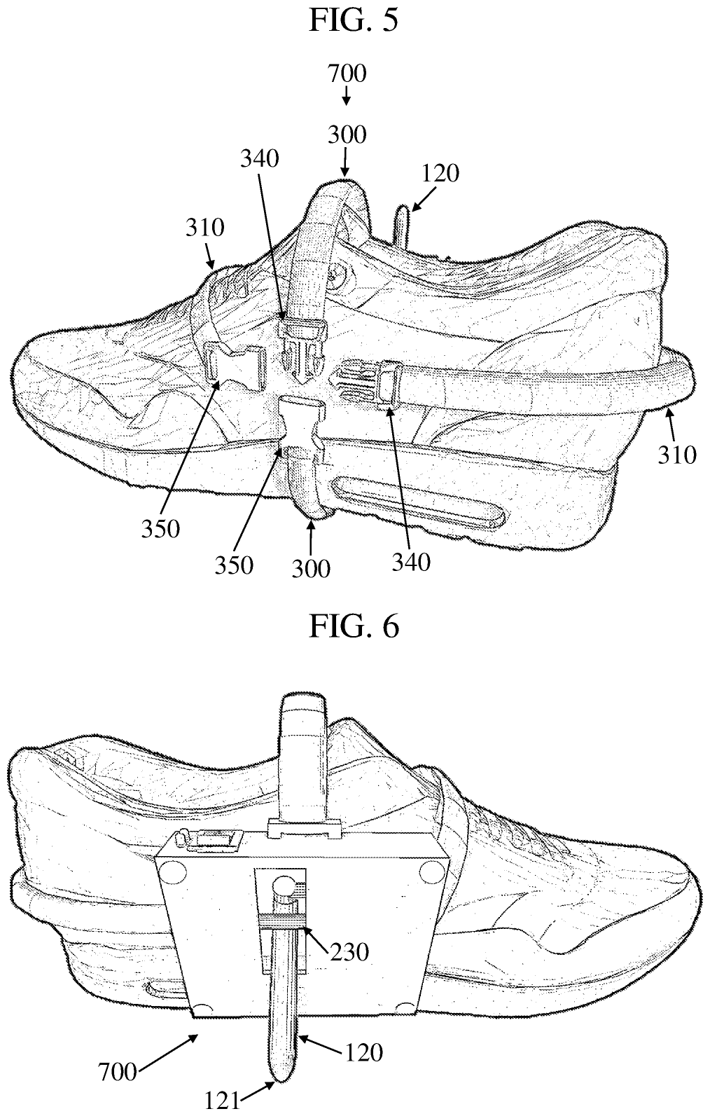

Shoe balancing device 700 features a housing 100, which may have a square or a rectangular shape, as illustrated, configured for removable attachment to a shoe. Such removable attachment can be accomplished via an attachment mechanism, for example, including attachment straps 300 and 310 configured to attach device 700 to the lateral side (the outer side) of the shoe and to the upper front and the rear (heel area) of the shoe, respectively (see also FIG. 5). Straps 300, 310 may be made of a stretchy material to help secure device 700 tightly in place.

Device 700 includes a stabilizing/balancing rod 120, configured for movement from an undeployed state (as in FIG. 1 and FIG. 2) to a deployed state (as in FIG. 6). Housing 100 includes a lid 240, for enclosing internal components (described below), and is configured to allow access to the inner components of device 700. Lid 240 may be attached to the remaining portion of the housing by any suitable fasteners, such as by screws 250 received by corresponding threads 260 (FIG. 4). Rod 120 may be made from any kind of rigid material (e.g., metals) and may be coated with rubber or the like to improve a stable interface between the free end 121 of the rod and the floor/ground or other surface. Device 700 may also include a solenoid pin 230 (FIG. 6) configured to reversibly lock the stabilization rod 120 in place after deployment thereof.

FIG. 2 shows optional battery charge indicators, exemplified by a green LED 160 and a red LED 161, operably connected to a hardware board 180 (FIG. 4). LEDs 160 and 161 provide indication to the user of the need to charge a battery 130 (e.g., a 3.7 Volt rechargeable lithium-ion polymer battery) chargeable via a charging port connector 140, which may be for example, a USB type-C charging port connector or micro-USB charging port connector (see also FIG. 4). Charging port connector 140 may be protected by a USB cover 145. The device may be turned on and off via a button 170.

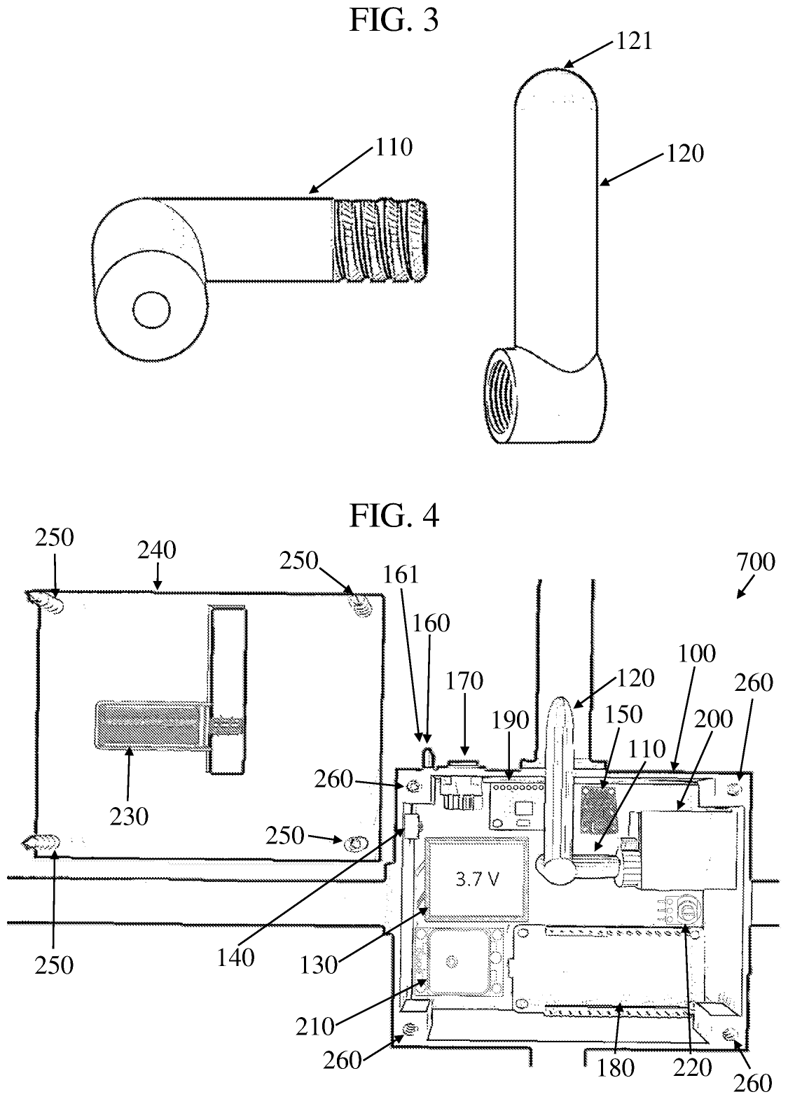

FIG. 3 shows internal components of a shoe balancing assembly of shoe balancing device 700, namely, stabilization rod 120 and a stabilization rod connector 110, which connects the stabilization rod to a servo motor 200 (FIG. 4). Housing 100 includes an opening therein through which rod connector 110 protrudes. In some examples, rod 120 is designed to be weaker than motor 200 and the connector 110 so that in an event of a significant force, the rod 120 will be replaceable (e.g., by threading a new rod 120 to the connector 110) and in order to protect the motor 200.

FIG. 4 shows previously described components as well as additional internal components of device 700. These internal components include a voltage-boost converter 150 (e.g., a mini DC-DC boost converter 0.9V-5V to 5V, 600 mA) in order to increase the voltage of battery 130 from 3.7V to 5V; a hardware board 180 (e.g. an open-source hardware board, such as an ESP32 hardware board) connected to battery 130, and configured to act as the operation control center; an accelerometer sensor 190 (e.g. a GY-521 MPU-6050 3 Axis gyroscope and 3 Axis Accelerometer Module), connected to servo motor 200 and configured to detect an imbalance state, in particular beyond a predetermined threshold; a GPS module 210, also connected to the hardware board, and configured to provide a location indication; and a potentiometer 220 (e.g., a 10K OHM linear taper rotary potentiometer), to read values from hardware board 180 and to activate (green) LED 160 when there is sufficient battery power and (red) LED 161 when the battery is weak. Device 700 may also include a solenoid pin 230 (FIG. 6) of a solenoid (not shown) to reversibly lock rod 120 in place.

FIG. 5 illustrates exemplary attachment portions of attachment straps 300 and 310, namely buckles composed of male and female buckle portions 340 and 350, for reversibly closing straps 300 and 310, so as to reversibly attach device 700 to the shoe. The attachment portions of straps 300 and 310 may alternatively be constituted by hook-and-loop fasteners.

Device 700 may further include a vibration or an audible warning mechanism (not shown) such that upon tilting thereof beyond a given angle, a vibration or a sound is emitted to provide an early warning to the wearer about a potential fall.

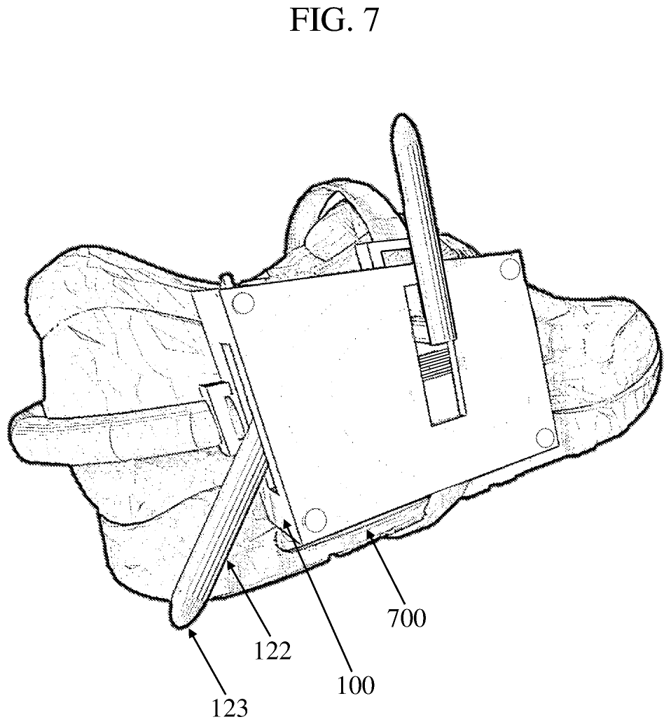

FIG. 7 illustrates device 700 also including a rear stabilization rod 122 having a free end 123 that extends outwardly from a rear wall of housing 100. Rear stabilization rod 122 is configured to assist in preventing backward falls and/or in preventing sideward-backward falls. When accelerometer and/or gyroscope sensor 190 detects a backward tilt, rear stabilization rod 122 rotates backwards (as shown in the figure) via its respective motor 200, which may be a separate motor or the same motor that actuates stabilization rod 120 and its respective solenoid pin (not shown) that locks rear stabilization rod 122 in the deployed state.

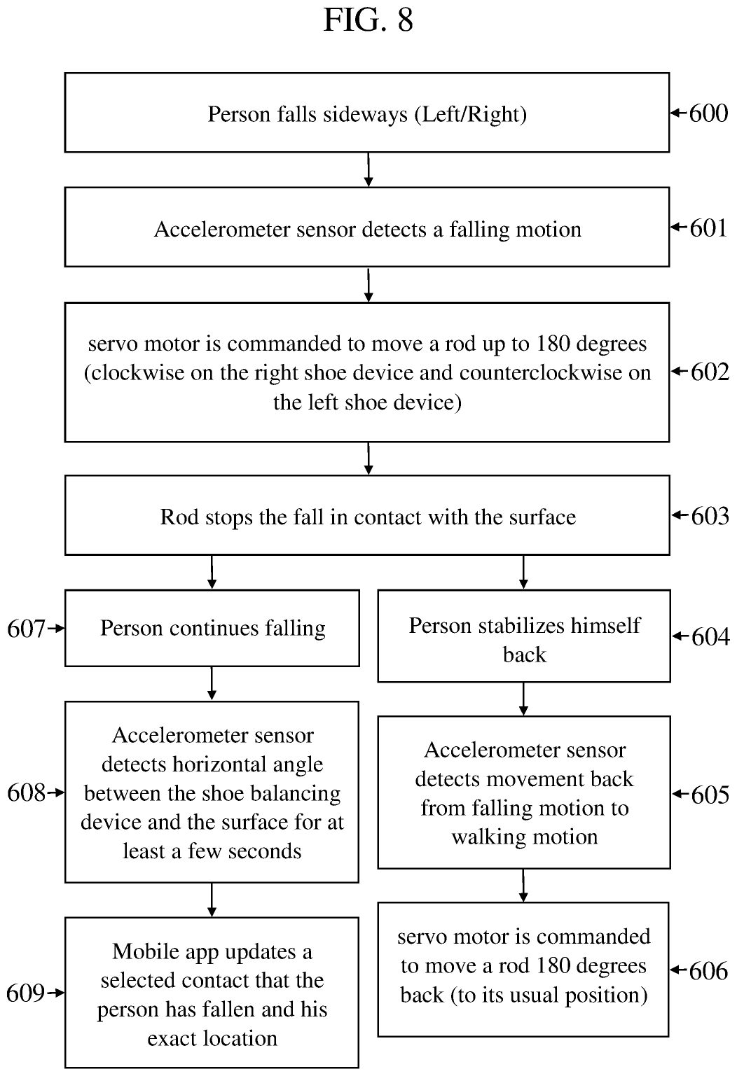

Operation of shoe balancing device 700 is exemplarily depicted in FIG. 8, namely deploying the device from a default undeployed state (FIGS. 1 and 2) to a deployed state (FIG. 6). If a wearer is leaning sideways to a situation (step 600) where device 700, via accelerometer and/or gyroscope sensor 190, determines a fall may be imminent (step 601), the device is activated, via accelerometer and/or gyroscope sensor 190, and appropriate steps are taken (steps 602-609).

In other words, a method of fall prevention or operation of the shoe balancing device includes, in a case of the wearer falling sideways 600, detecting the falling 601; and instructing stabilizing rod 120 to deploy, i.e., moving the stabilizing rod downward to engage the floor/ground (surface) 602, 603. Deployment of rod 120 is performed by activation of motor 200 to rotate, translate, or otherwise move, the free end 121 of the rod downward.

If accelerometer and/or gyroscope sensor 190 determines that the falling has been prevented (i.e., the wearer is stabilized 604), then stabilizing/balancing rod 120 is returned to its undeployed default/initial state (step 605). However, if the wearer continues to fall (step 607), as determined by accelerometer and/or gyroscope sensor 190 detecting an angle beyond a threshold angle (in particular, generally horizontal) with respect to the surface under the wearer (step 608) for a predetermined time that would indicate that the fall has not been prevented, i.e. the wearer has fallen, a signal is sent (step 609) from hardware board 180, which may connect via a Bluetooth or Wi-Fi, for example, to a mobile software App (application) of the wearer. The data may then be transmitted to a monitoring service (such as an aid/caregiving service or individual). The data transmitted may include an indication of the wearer's location, as determined by GPS module 210. In some examples, device 700 includes a timer, which can be incorporated in one or more of the afore-mentioned components, or be a separate component, to indicate the time that has lapsed since the fall, thereby providing an indication of the seriousness of the fall and possibly the condition of the wearer. Optionally, and in cases wherein the wearer uses a pair of the herein devices (i.e., one device attached to the right shoe and another to the left shoe), the dedicated mobile software App may be programmed to communicate with only one of the devices (via Bluetooth or Wi-Fi). In such cases, the hardware board 180 of one device may incorporate a protocol algorithm that allows the App to communicate with the hardware board 180 of the other device. Such a configuration allows the devices to share information if necessary (for example the ESP32 hardware board may incorporate an algorithm protocol named ESP-NOW which enables multiple devices to communicate with one another without using Wi-Fi).

Thus, a device and method are herein disclosed for providing support to a falling person. The device and method can help prevent a fall, and in a case where a fall has regardless occurred, the device/method provide for signaling help.

As used herein the terms ‘a’ and ‘an’ may mean ‘one’ or ‘more than one’. All ranges disclosed herein include the endpoints. The use of the term “or” shall be construed to mean “and/or” unless the specific context indicates otherwise. Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The term ‘consisting essentially of’ as used herein means limited to the specified elements and those that do not materially affect the basic and novel characteristic(s) of the claimed presently disclosed subject matter. Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed presently disclosed subject matter.

Throughout this disclosure, a numerical value of a parameter, feature, characteristic, object, or dimension, may be stated or described in terms of a numerical range format. Such a numerical range format, as used herein, illustrates implementation of some exemplary embodiments of the presently disclosed subject matter, and does not inflexibly limit the scope of the exemplary embodiments of the presently disclosed subject matter. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, in some embodiments, refers to ±30% of the stated numerical value. In further embodiments, the term refers to ±20% of the stated numerical value. In yet further embodiments, the term refers to ±10% of the stated numerical value.

It should be understood that the above description is merely exemplary and various embodiments of the present presently disclosed subject matter may be devised, mutatis mutandis, and that the features described in the above-described embodiments, and those not described herein, may be used separately or in any suitable combination; and the presently disclosed subject matter can be devised in accordance with embodiments not necessarily described above.

1-24. (canceled)

25. A shoe balancing device comprising:
a housing configured to be attached to a shoe of a wearer;
a power source to power the device;
an accelerometer and/or gyroscope sensor configured to determine an imbalance state of the device;
a hardware board configured to act as the operation control center of the device;
a motor configured to be activated by the hardware board if the accelerometer and/or gyroscope sensor determines an imbalance state beyond a predetermined threshold; and
a stabilization rod operably connected to the motor so as to be moveable from a default undeployed state to a deployed state, the deployed state being a position in contact with a surface underneath the wearer's location, wherein when a wearer of the shoe begins to fall the device is actuated.

26. The device of claim 25, comprising straps to attach the housing to the shoe.

27. The device of claim 25, further comprising a GPS module to provide location information of the wearer.

28. The device of claim 25, wherein the motor is configured to move the stabilization rod downwardly toward the surface to thereby apply a resistance force thereto.

29. The device of claim 25, wherein the motor is configured to linearly move the stabilization rod.

30. The device of claim 25, further comprising a solenoid pin configured to reversibly lock the stabilization rod in place after deployment thereof.

31. The device of claim 25, wherein the device is configured to be positioned on a right shoe, a left shoe, or both shoes.

32. The device of claim 25, wherein the device is configured to be positioned on the lateral facet of a wearer shoe.

33. The device of claim 25, wherein the device is configured to stabilize the wearer and prevent falling thereof.

34. The device of claim 25, further comprising an alarm mechanism comprising a vibration and/or an audible warning to allow for an advance warning to the wearer about a potential fall.

35. The device of claim 25, wherein the stabilization rod is configured to revert to the undeployed state when the wearer is vertically stabilized.

36. The device of claim 25, wherein the threshold is at least 10 degrees movement of the wearer toward the ground/floor.

37. The device of claim 25, wherein the stabilization rod is operably connected to the motor via a stabilization rod connector.

38. The device of claim 25, wherein the stabilization rod outwardly protrudes from a side wall or a rear wall of the housing when the device is actuated.

39. A method of preventing or indicating a fall of a wearer of a shoe balancing device, the method comprising:
determining if the shoe balancing device is at an angle beyond a predetermined threshold;
activating a stabilizing rod, if said threshold is surpassed, to move from a non-deployed default state to a deployed state, wherein a free end of the stabilizing rod is moved to a position that interfaces with the surface underneath the wearer.

40. The method of claim 39, wherein the stabilizing rod is linearly moved by the rod toward the ground/floor to stabilize the wearer.

41. The method of claim 39, wherein if the threshold is surpassed by a predetermined period of time, a signal is sent to a telephone of another person and/or to a monitoring service.

42. The method of claim 39, wherein the predetermined period of time is 10 seconds or above.

43. The method of claim 39, comprising providing a location of the device.

44. The method of claim 39, wherein the threshold is at least 10 degrees movement of the wearer toward the ground/floor.
